(19) United States
(12) Patent Application Publication
(10) Pub. No.: US 2008/0183299 A1
(43) Pub. Date: Jul. 31, 2008

(54) URETERAL STENT WITH AXIAL AND RADIAL VARIABILITY

(75) Inventors: Manoj Monga, Minneapolis, MN (US); William K. Durfee, Edina, MN (US)

Correspondence Address: BRINKS HOFER GILSON & LIONE/CHICAGO/COOK, PO BOX 10395, CHICAGO, IL, 60610, US

(73) Assignee: Vance Products Inc., d/b/a/ Cook Urological Inc., Spencer, IL (US)

(21) Appl. No.: 11/960,243

(22) Filed: Dec. 19, 2007

Related U.S. Application Data
(63) Continuation of application No. PCT/US2006/023829, filed on Jun. 20, 2006.
(60) Provisional application No. 60/692,742, filed on Jun. 21, 2005.

(51) Int. Cl. A61F 2/04

(52) U.S. Cl. 623/23.66; 623/23.7

(57) ABSTRACT

A device is provided that includes an implantable conduit with first and second ends and a first retainer coupled to the first end by a first joint. A second retainer is coupled to the second end by a second joint, wherein the first and second joints are each configured to allow movement of the implantable conduit relative to the respective first and second retainers.

### CLAIM OR PRIORITY

Benefit of priority is hereby claimed to U.S. Provisional Patent Application Ser. No. 60/692,742, filed on Jun. 21, 2005, which application is herein incorporated by reference.

### TECHNICAL FIELD

This document pertains generally to medical devices, and more particularly, but not by way of limitation, to a ureteral stent with axial and radial variability.

### BACKGROUND

The ureter is a channel that drains urine from the kidney to the bladder. A ureteral stent provides tract drainage and relief for ureteral obstructions which leads to pain in the kidney. An obstruction can be caused by a stone, stricture or a tumor.

In some cases, the stent is associated with pain arising from physical movement, respiration and bladder contractions and expansions of the patient.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1A illustrates a ureteral stent with a co-axial introducer.

FIG. 1B illustrates a ureteral stent with curled ends.

FIGS. 2A-2H illustrate flexible couplings on a ureteral stent.

FIG. 3 illustrates a flowchart for manufacturing a stent.

FIG. 4A illustrates a conduit segment having a plurality of rectangular holes in an array.

FIG. 4B illustrates a view of expanded elastomeric material.

FIG. 4C illustrates a view of a conduit having circular holes distributed proximate the ends.

FIG. 4D illustrates a view of a conduit having helical ends.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

The etiology of pain associated with an implantable ureter stent is unclear. Possibilities include irritation of the kidney, irritation of the bladder or irritation of both the kidney and bladder, or other sources such as kidney pain caused by reflux of urine up the stent to the kidney.

An implantable device according to one embodiment of the present subject matter includes a stent having retention structures at each end and at least one retention structure allows independent movement relative to the stent. As such, the stent can remain stationary in the ureter while the retention structures translate with movement of the kidney and bladder. One example of the device provides flexible length to decrease movement of the stent during patient movement, bladder movement and respiration.

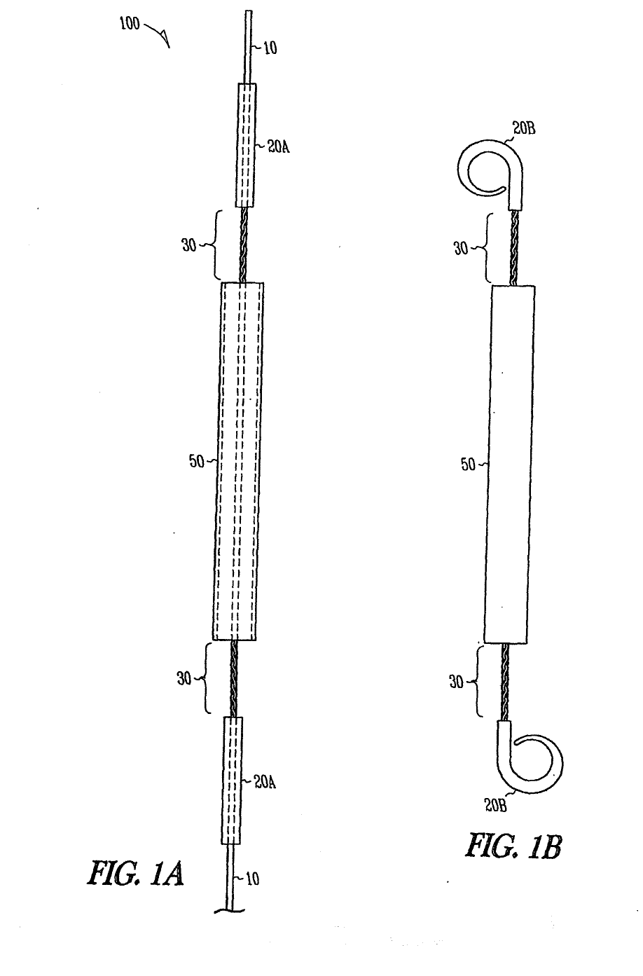

FIG. 1 illustrates implantable device 100 having co-axial introducer 10. In the figure, device 100 is made of bio-compatible materials and includes stent 50 coupled to retainers 20A by flexible joints 30. Stent 50 includes a stent lumen, depicted by the hidden lines representing a wall thickness, through which introducer 10 is disposed. In addition, retainers 20A each include a retainer lumen through which introducer 10 is also disposed.

Stent 50 includes tubular materials such as molded or extruded silicone or polyurethane, polyamide, polyvinyl chloride or other polymer having a relatively fixed length between flexible joints 30.

In the example illustrated, retainers 20A each include flexible material such as silicone or polyurethane, polyamide, polyvinyl chloride other polymer or metal. The material naturally returns to a curled or coiled configuration as shown in FIG. 1B at retainers 20B and when reinforced with internal introducer 10, retainers 20A is held straight to facilitate placement in the ureter. The illustrated configuration of the curled retainers is sometimes referred to as a “double-J.”

Introducer 10 can include a rigid or flexible wire or elongate polymer structure that facilitates introduction of the device in the ureter. Introducer 10 can also be referred to as a guide wire.

Flexible joints 30 each include a flexible material such as silicone or polyurethane, polyamide, polyvinyl chloride or other polymer or metal. The flexible joints are configured to allow relative movement or variability between each of retainers 20A and stent 50. For example, movement can occur along or about the longitudinal axis of introducer 10. As such, movement can be in an axial direction, a radial direction or any combination of both axial and radial movements, with respect to the longitudinal axis. Flexible joints 30 are disposed at each end of stent 50 and provide a transition between retainers 20A and stent 50.

In FIG. 1B, retainers 20B are in a relaxed or unloaded condition and have a coiled profile. When implanted, one retainer 20B is disposed within a bladder and one retainer 20B is disposed in a kidney. The coiled profile serves to maintain stent 50 within the ureter coupling the bladder and kidney.

Retainers other than the curled type are also contemplated. For example, a fluted or flared structure having an outer cross-sectional dimension greater than an inner cross-sectional dimension can also serve as the retainer.

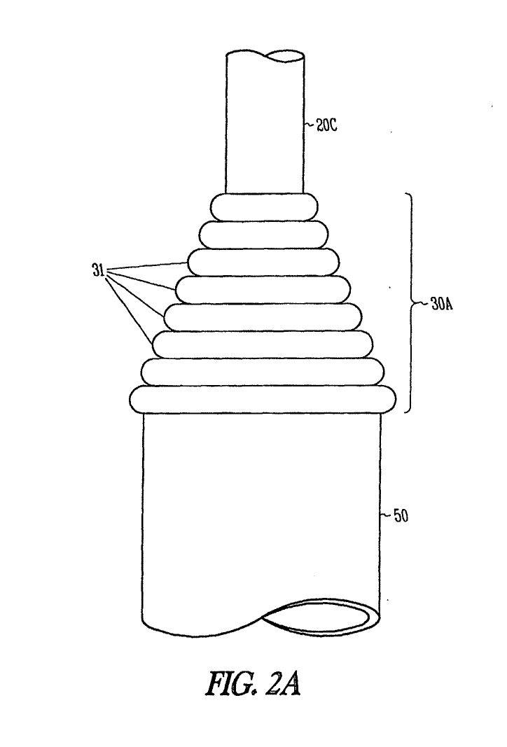

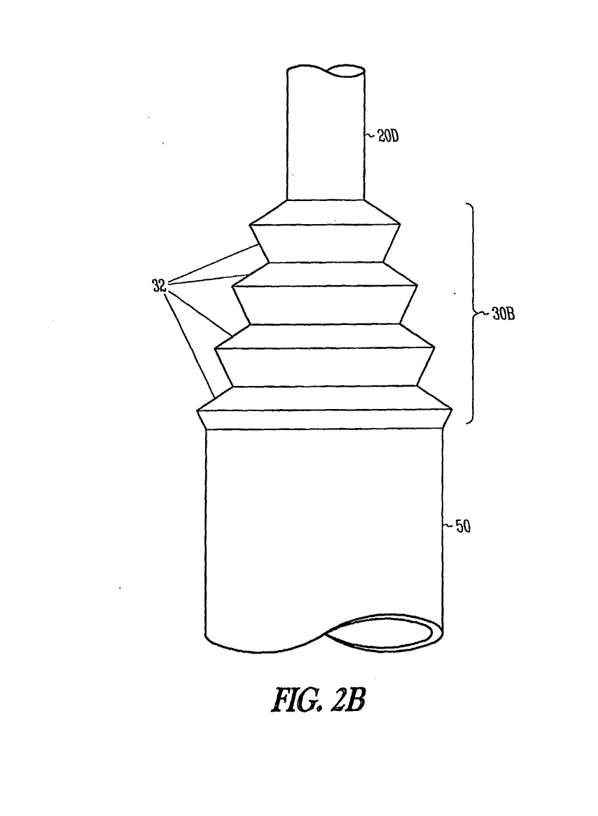

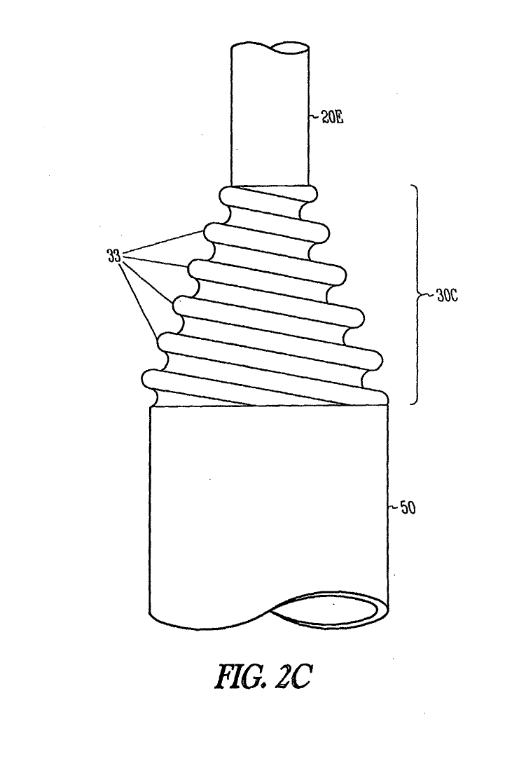

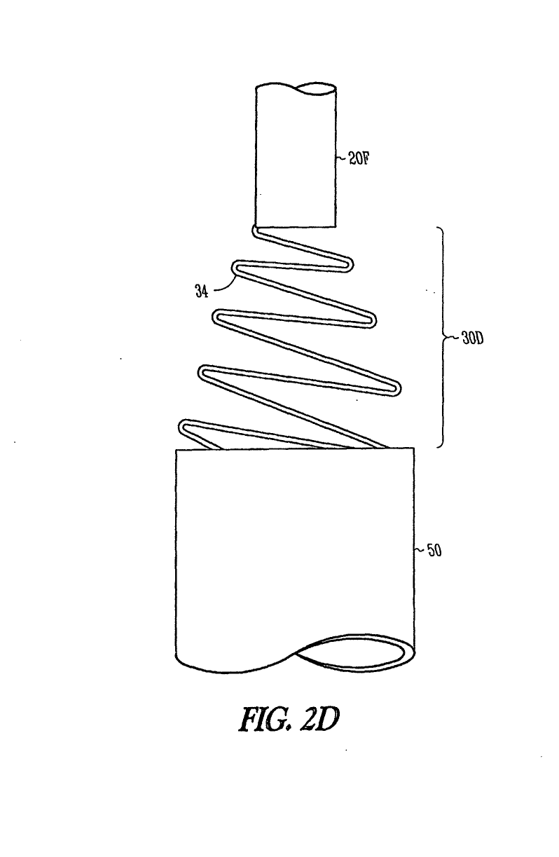

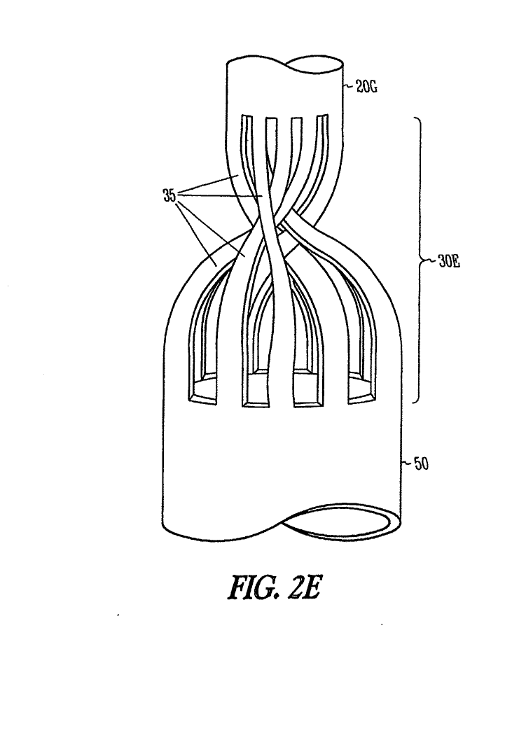

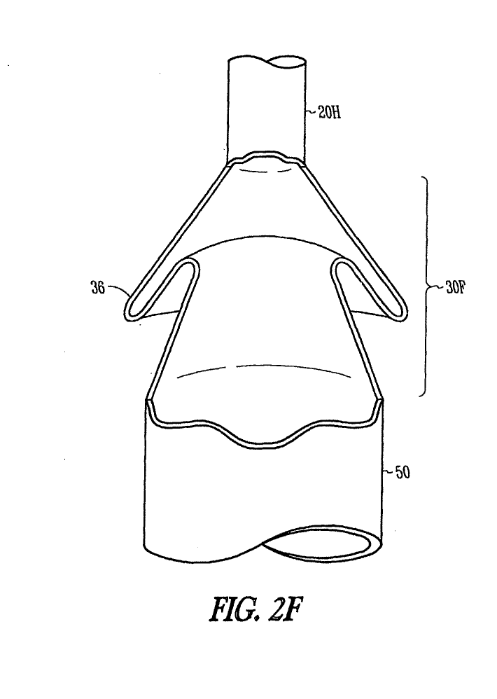

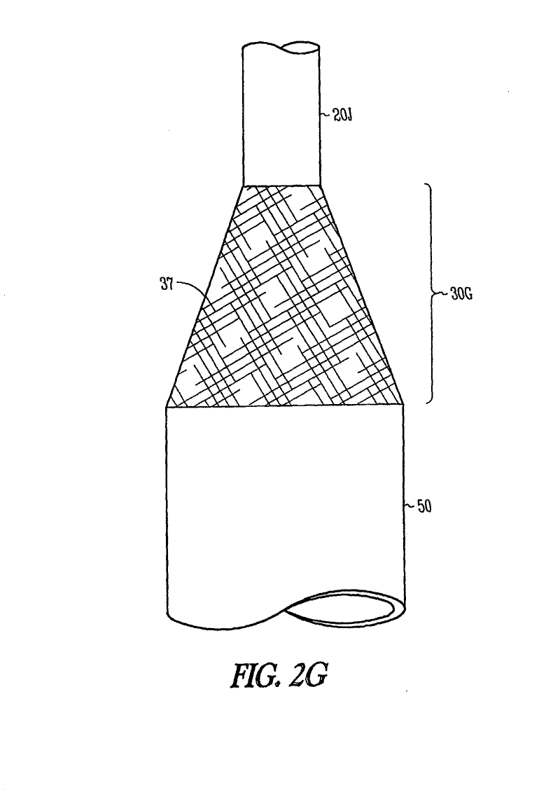

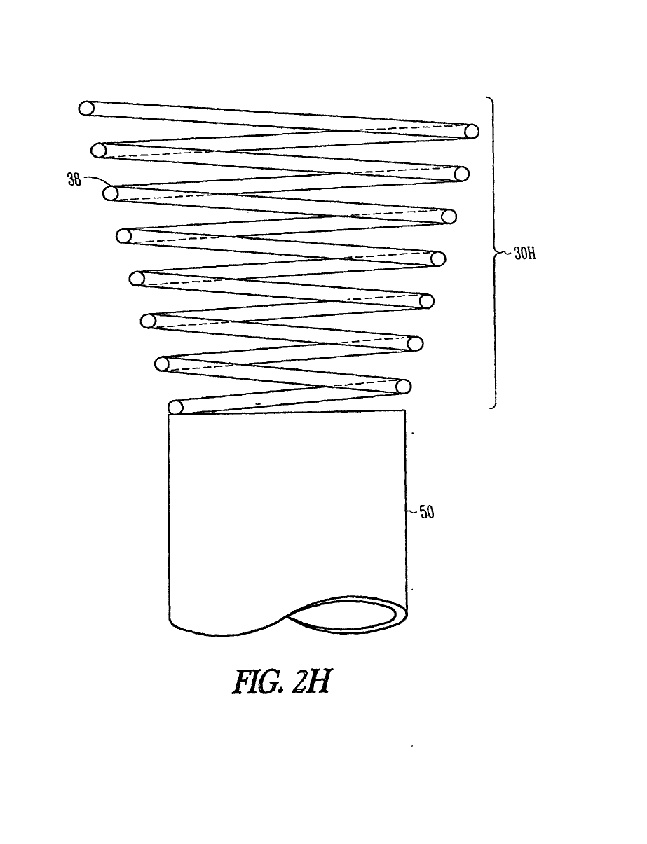

FIG. 2A illustrates a view of a single flexible joint 30A coupled between stent 50 and retainer 20C. In the figure, joint 30A includes a plurality of axially aligned annular rings or segments 31 arranged in a bellows configuration which allows relative movement between adjacent segments 31.

Joint 30A includes a molded or formed tubular material wherein each segment 31 is integral with an adjacent segment 31. In various examples, the coupling between retainer 20C and joint 30A as well as the coupling between stent 50 and joint 30A includes a bond or a mechanical fastener. In one example, at least one of the coupling between retainer 20C and joint 30A and the coupling between stent 50 and joint 30A is contiguous.

In the figure, the lumen of stent 50 is visible. In various examples, retainer 20C includes a solid (as illustrated) or fluid conducting structure. In various examples, flexible joint 30A includes a solid or fluid conducting structure.

FIG. 2B illustrates a single flexible joint 30B coupled between stent 50 and retainer 20D. In the figure, joint 30B includes a plurality of axially aligned pleated segments 32 in a bellows configuration which allows relative movement between adjacent segments 32.

Joint 30B includes a molded or formed tubular material wherein each segment 32 is integral with an adjacent segment 32. In various examples, the coupling between retainer 20D and joint 30B as well as the coupling between stent 50 and joint 30B includes a bond or a mechanical fastener. In one example, at least one of the coupling between retainer 20D and joint 30B and the coupling between stent 50 and joint 30B is contiguous.

In the figure, the lumen of stent 50 is visible. In various examples, retainer 20D includes a solid (as illustrated) or fluid conducting structure. In various examples, flexible joint 301B includes a solid or fluid conducting structure.

FIG. 2C illustrates a view of a single flexible joint 30C coupled between stent 50 and retainer 20E. In the figure, joint 30C includes a helically wound bellows having a continuous spiral of segments 33. Joint 30C allows relative movement between adjacent segments 33 as well as between retainer 20E and stent 50.

Joint 30C includes a molded or formed tubular material wherein each segment 33 is integral with an adjacent segment 33. In various examples, the coupling between retainer 20E and joint 30C as well as the coupling between stent 50 and joint 30C includes a bond or a mechanical fastener. In one example, at least one of the coupling between retainer 20C and joint 30C and the coupling between stent 50 and joint 30C is contiguous.

In the figure, the lumen of stent 50 is visible. In various examples, retainer 20E includes a solid (as illustrated) or fluid conducting structure. In various examples, flexible joint 30C includes a solid or fluid conducting structure.

FIG. 2D illustrates a view of a single flexible joint 30D coupled between stent 50 and retainer 20F. In the figure, joint 30D includes helical spring 34 having a conical profile which allows relative movement between retainer 20F and stent 50.

Spring 34, in various examples, includes a wire or polymer material formed by drawing, molding or winding. In various examples, the coupling between retainer 20F and joint 30D as well as the coupling between stent 50 and joint 30D includes a bond or a mechanical fastener. In one example, at least one of the coupling between retainer 20F and joint 30D and the coupling between stent 50 and joint 30D is contiguous.

In the figure, the lumen of stent 50 is visible. In various examples, retainer 20F includes a solid (as illustrated) or fluid conducting structure. Flexible joint 30D is illustrated as a coil spring in which fluid is free to pass between adjacent windings as well as axially.

FIG. 2E illustrates a view of a single flexible joint 30E coupled between stent 50 and retainer 20G. In the figure, joint 30E includes a plurality of discrete flexible woven filaments 35. Each filament 35 is interlaced with other filaments 35 to provide a woven structure that maintains an internal diameter which increases slightly when retainer 20G is drawn near stent 50 and decreases slightly when retainer 20G is moved apart from stent 50.

A variety of weaves can be used for joint 30E. For example, a braid or looped weave provides flexibility and dimensional stability to maintain a relatively constant flow channel through the longitudinal axis of joint 30E. In addition, a loose weave allows fluid to pass through the interstitial space between individual filaments 35. In the figure, the filaments are shown to be ribbon-like and have a substantially rectangular cross section, however other cross sections are also contemplated, including, for example, round or oval profiles.

Filaments 35 can be drawn or molded. In various examples, the coupling between retainer 20G and joint 30E as well as the coupling between stent 50 and joint 30E includes a bond or a mechanical fastener. In one example, at least one of the coupling between retainer 20G and joint 30E and the coupling between stent 50 and joint 30E is contiguous.

In the figure, the lumen of stent 50 is visible. In various examples, retainer 20G includes a solid (as illustrated) or fluid conducting structure. In various examples, flexible joint 30E includes a solid or fluid conducting structure.

FIG. 2F illustrates a cut-away view of a single flexible joint 30F coupled between stent 50 and retainer 20H. In the figure, joint 30F includes an intussuscepted joint where a thin section of material 36 is folded or inverted inwards into a first segment of material 36. The intussuscepted joint allows relative movement between adjacent portions of material 36 and thus, allow movement between retainer 20H and stent 50.

Joint 30F includes a molded or formed tubular material having a thin wall section. In various examples, the coupling between retainer 20H and joint 30F as well as the coupling between stent 50 and joint 30F includes a bond or a mechanical fastener. In one example, at least one of the coupling between retainer 20H and joint 30F and the coupling between stent 50 and joint 30F is contiguous.

In the figure, the lumens of stent 50 and retainer 20H are visible. In various examples, retainer 20H includes a solid or a fluid conducting structure. In various examples, flexible joint 30F includes a solid or fluid conducting structure (as illustrated).

FIG. 2G illustrates a view of a single flexible joint 30G coupled between stent 50 and retainer 20J. In the figure, joint 30G includes non-woven material 37 having a plurality of fibrous elements intertwined in a mesh that allows relative movement between retainer 20J and stent 50. Material 37, in various examples, includes a polymer or extruded fibers that are blended together in manner that allows dimensional flexibility.

Joint 30G includes a molded, formed or spun section of material 37. In various examples, the coupling between retainer 20J and joint 30G as well as the coupling between stent 50 and joint 30G includes a bond or a mechanical fastener. In one example, at least one of the coupling between retainer 20J and joint 30G and the coupling between stent 50 and joint 30G is contiguous.

In the figure, the lumen of stent 50 is visible. In various examples, retainer 20J includes a solid (as illustrated) or fluid conducting structure. In various examples, flexible joint 30G includes a solid or fluid conducting structure.

FIG. 2H illustrates a flexible joint integrated with a retainer. In the figure, stent 50 is coupled to joint 30H configured to retain the device in the ureter and also provide relative flexibility. Joint 30H includes spring 38 having a fluted or flared profile to prevent inadvertent extraction from the kidney or bladder. Joint 30H is placed in position using an external sheath or an internal guide that holds spring 38 in a retracted position. Joint 30H is bonded or mechanically fastened to stent 50. Suitable materials for spring 38 include various polymers and metals.

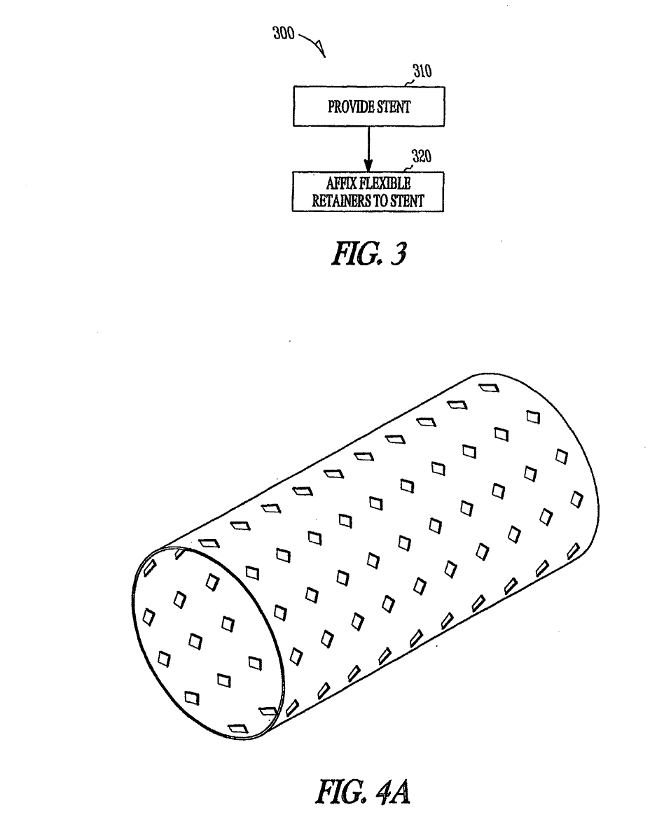

FIG. 3 illustrates method 300 for fabricating device 100. At 310, a stent is provided. The stent includes a tubular structure having one or more fluid conducting lumens. At 320, a flexible retainer is affixed to the stent. The retainer can be affixed as an integral part of the manufacturing process by which the stent is provided or affixed in a separate bonding or mechanical fastening procedure. In various examples, the retainer includes a discrete flexible joint, as shown in FIGS. 2A-2G, or the joint and retainer are integral as illustrated in FIG. 2H. In one example, both a first and second retainer are flexibly mounted to the stent.

The variability between retainers 20 (sometimes referred to as a retention structure) and stent 50 (sometimes referred to as a medial structure) of the present subject matter may ameliorate pain associated with an implantable device.

In addition, the present subject matter can be implemented in other implantable devices configured to allow relative motion between a retention structure and a medial structure. Furthermore, the retention structures of the present subject matter can be disposed at one or more locations along a length of a linear structure. For example, a first retention structure can be disposed at a first end of the implantable stent and a second retention structure can be disposed at a position along the length of the stent rather than at the second end of the stent. As such, a portion of the stent extends beyond the position of the second retention structure.

### Alternative Examples

In addition to those examples illustrated and described above, other structure and methods are also contemplated. For instance, one example includes an implantable conduit having a fixed length and wherein the conduit includes a fluid conducting lumen. A retention device is coupled to one or both ends of the conduit, and in one example, the retention device has a variable length. In one example, the retention device includes a channel that is in fluid communication with the lumen of the conduit. In one example, the lumen terminates at an exit port which does not include the retention structure and as such, the retention structure does not include a fluid channel. In addition to implantation in a ureter, the present subject matter can be used in other ducts including a drain or a fluid channel.

In one example, the implantable conduit is coupled to a woven mesh or other flexible joint at either one or both ends. In an example having a single flexible joint coupled to the conduit and the retainer, the non-flexible retainer is disposed in the kidney and the flexible retainer is disposed in the bladder.

In one example, the device includes a woven pleated soft polymer spring mesh interspersed between both the proximal and distal retention structures and the shaft of the stent. The mesh provides a flexible joint. The flexible joint allows both expansion and contraction with movement of the patient. In one example, the mesh includes a polymer such as silicone or polyurethane. Other compliant materials are also contemplated.

It is believed that a flexible joint can ameliorate significant pain in the flank and/or bladder arising from movement of the stent as the patient changes position. The present subject matter may reduce ureteral stent movement and may reduce movement of the coiled segments. The flexible joint is configured to elongate when stretched and also simultaneous decrease an outside diameter of the mesh. This ability to elongate, then return to a baseline length allows the stent to move in a bellows-like manner or in a manner similar to a popular children's novelty device sometimes referred to as a Chinese finger trap. In various examples, the mesh includes a polymer of the type sometimes used for fabricating stents and is resistant to encrustation.

In one example, the longitudinal movement of the stent with anatomic movement of the patient is between 2 and 5 cm. The stent movement can include bowing, moving and sliding. The stent is configured for movement relative to the kidney and bladder.

In one example, the flexible joint includes a helix formed in a portion of the stent. Other examples are also contemplated, including for instance, a scaffold where the fluid passes between the structure of the scaffold and not exclusively in a longitudinal direction.

Other combinations or configurations for the flexible joint are also contemplated. For example, a woven pleat or spiral design can be used. The spiral design allows for greater expansion and simplicity and may facilitate implantation. A variety of flexible joints are illustrated and described herein. One flexible joint may differ from another flexible joint of the same device. For example, a coil spring type retainer device may be disposed on a first end of a stent and a woven joint may be disposed on a second end of the same stent. In addition, the joints may differ in terms of the types of materials, sizes and configuration. In one example, a shape memory material is used for the flexible joint or for the retainer. Shape memory alloys include temperature sensitive polymers as well as nickel alloys.

In one example, the flexible joint includes a sleeve structure having two or more nested tubes that allow relative movement there between. Although the figures illustrate a retainer having a diameter smaller than the stent, other configurations are also contemplated. For example, the retainer and stent may have the same diameter or one may be smaller than the other. In addition, neither the stent nor the retainer are necessarily circular in cross section. Rectangles and flat materials are also contemplated.

In various examples, an flexible device is formed by perforating and stretching in a longitudinal or axial direction. In one example, a pattern of slots or holes are formed in a wall section of an elastomeric conduit and the conduit is clamped and stretched in a transverse direction to form an elastic lattice structure. The lattice structure is flexible and allows changes in length and alignment relative to the main portion of the conduit. The lattice structure can be localized near one or both ends of the conduit. In one example, the lattice structure is distributed along the entire length of the conduit. In one example, the lattice structure is configured to include holes of a higher density near the ends of the conduit.

The lattice structure can be formed of overlapping or staggered arrangement of holes formed in the elastomeric material. The holes can be of identical or different sizes and have sharp or curved ends to form a lattice having a series of longitudinally extending stretchable portions. The elastomeric material can be superelastic or semi-rigid and the lattice is formed by stretching to a strain value beyond their yield point.

The elastic property of elastomeric materials, when configured in a lattice, provides on-demand lengthening of a ureteral stent to improve patient comfort.

The flexibility of the lattice structure can be selected based on the spacing, arrangement, numerosity, alignment, shape, and density of holes as well as the selection of the material for the elastomeric material. In one example, the resiliency allows a variation in length of 5-6 cm. In addition to axial flexibility, a particular lattice configuration can be selected to provide radial flexibility.

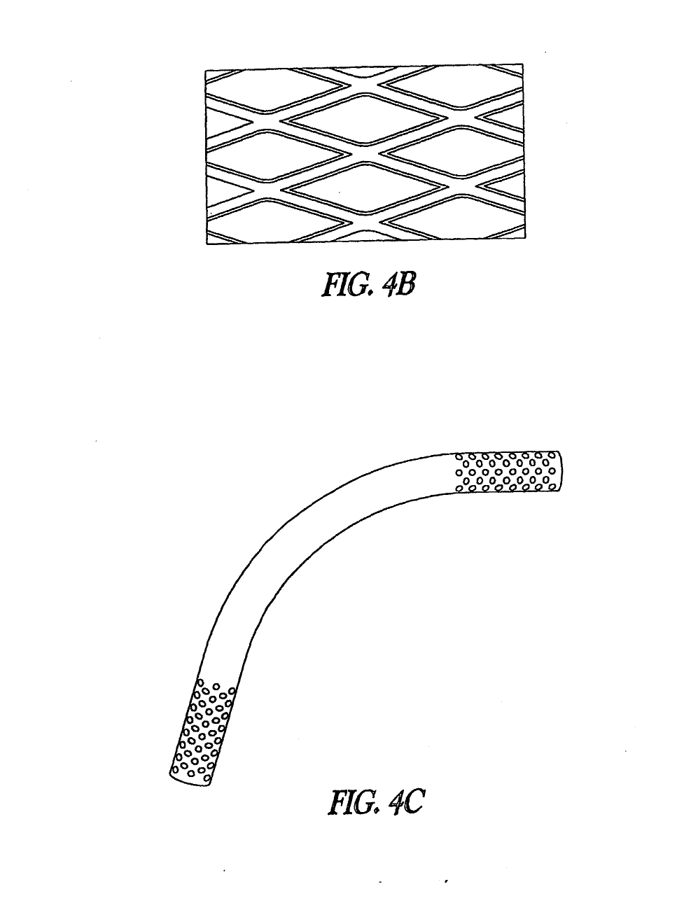

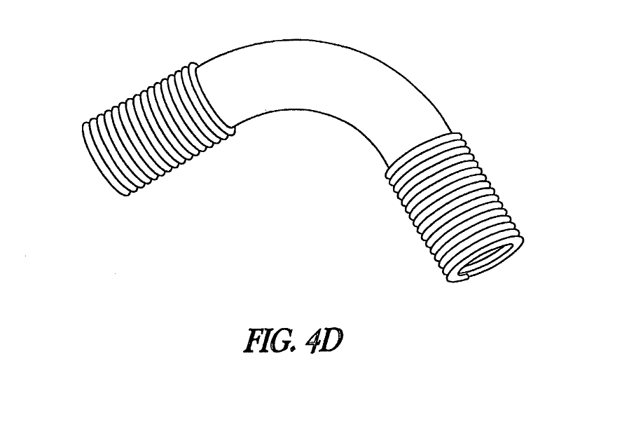

The elastomeric material can include silicone or other polymer. In one example, the elastomeric material includes a silicone tubing of ⅜″ outside diameter and 3/16″ inside diameter with a hole configuration of 4.242 mm spacing. The holes can be of an arbitrary shape and in one example, are configured in a diamond-shaped pattern, as illustrated in FIG. 4A. The portion of a conduit segment shown includes rectangular holes in a uniform distribution.

In one example, the stent includes a retention coil or other structure that absorbs forces associated with patient movement to improve patient comfort.

In one example, the stent includes expanded portions having small perforations (holes) in the tubular conduit. A force applied in an axial or radial direction causes expansion of the perforations. The perforations can include slits or holes where the holes are rectangular, faceted or circular in configuration. The perforations can be distributed in a uniform or random pattern with flexibility in a particular dimension determined by, among other things, the hole density. The perforations can be distributed about the periphery of a conduit or along a portion of the conduit. The perforations are formed in an elastomeric material. In one example, the holes return to their original configuration after removal of a deflection force. In one example, rectangular shaped holes are disposed on the conduit surface and pulled axially. The elasticity of the structure allows resilience.

FIG. 4B illustrates a portion of a conduit wall segment having a plurality of slit perforations. In the figure, the conduit wall segment is shown following application of an axial force used to expand the material, thus forming diamond-shaped holes.

FIG. 4C illustrates a portion of a conduit having circular shaped holes. The holes are disposed near the ends of a curved conduit in the form of a ureteral stent. The holes shown are of uniform diameter and are arranged on the surface of the conduit in a manner selected for particular flexibility.

FIG. 4D illustrates a curved conduit having ends configured in the form of helical springs. The helical springs shown have coils of uniform diameter and uniform pitch, however, it will be understood that non-uniform diameters and pitch can also be selected to provide a desired spring performance.

In addition to considerations of flexibility, the present subject matter includes a conduit configured to exhibit fluid flow characteristics having particular flow turbulence and propensity to encrust.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. A device comprising:
an implantable conduit having a first end and a second end;
a first retainer coupled to the first end by a first joint; and
a second retainer coupled to the second end by a second joint, wherein the first joint and the second joint are configured to allow movement of the implantable conduit relative to the first retainer and second retainer.

2. The device of claim 1 wherein at least one of the first retainer and the second retainer includes a retainer lumen and wherein the conduit includes a conduit lumen and further wherein the conduit lumen is coupled to the retainer lumen.

3. The device of claim 1 wherein at least one of the first retainer and the second retainer includes a perforated segment.

4. The device of claim 1 wherein at least one of the first retainer and the second retainer includes a spring.

5. The device of claim 1 wherein at least one of the first joint and the second joint includes a polymer.

6. The device of claim 5 wherein at least one of the first joint and the second joint includes a pleat.

7. The device of claim 1 wherein at least one of the first joint and the second joint includes a plurality of flexible segments.

8. A method comprising:
providing a first retainer flexibly coupled to a first end of an implantable conduit; and
providing a second retainer flexibly coupled to a second end of the implantable conduit wherein the first and second retainer allow relative movement of the implantable conduit.

9. The method of claim 8 wherein providing the first retainer includes forming an expandable joint.

10. The method of claim 8 wherein providing the first retainer includes forming a pleated joint.

11. The method of claim 8 wherein providing the first retainer includes forming a woven joint.

12. The method of claim 8 wherein providing the first retainer includes forming by molding.

13. A system comprising:
an implantable conduit having a fixed length between two ends along a longitudinal axis; and
a first terminal and second terminal, each terminal affixed to an end of the implantable conduit and wherein each terminal is configured for movement independent of the conduit.

14. The system of claim 13 wherein at least one terminal includes a perforated segment.

15. The system of claim 13 wherein at least one terminal includes a spring.

16. The system of claim 13 wherein at least one terminal includes a mesh.

17. The system of claim 13 wherein at least one terminal includes a pleated joint.

18. The system of claim 13 wherein at least one terminal includes an intussuscepted joint.

19. The system of claim 13 wherein at least one terminal includes a woven joint.

20. The system of claim 13 wherein at least one terminal includes a polymer.

21. The system of claim 13 wherein the conduit includes a polymer.

22. The system of claim 13 wherein at least one terminal is configured for longitudinal movement independent of the conduit.

23. The system of claim 13 wherein the conduit includes a conduit lumen and at least one terminal includes a terminal lumen and wherein the conduit lumen is in fluid communication with the terminal lumen.

24. An implantable device comprising a conduit having a flexible segment, the flexible segment having a lattice structure to allow variations in a dimension of the conduit.

25. The implantable device of claim 24 wherein the lattice structure includes a plurality of perforations disposed about a periphery of the conduit.

26. The implantable device of claim 24 formed of an elastic material.

27. The implantable device of claim 24 wherein the elastic material includes silicone.
